(19) United States
(12) Patent Application Publication
(10) Pub. No.: US 2004/0037868 A1
(43) Pub. Date: Feb. 26, 2004

(54) Select prebiotics for use in weight attenuation message and method of communication

(76) Inventors: Deanna M. Minich, Minneapolis, MN (US); Ellen M. Silva, Minnetonka, MN (US); Michael A. Helser, Plymouth, MN (US)

Correspondence Address: GENERAL MILLS, INC., P.O. BOX 1113, MINNEAPOLIS, MN, 55440, US

(21) Appl. No.: 10/224,121

(22) Filed: Aug. 20, 2002

(51) Int. Cl. A61K 47/00; A23L 1/00

(52) U.S. Cl. 424/439; 426/531

(57) ABSTRACT

The present invention relates to the inclusion of certain prebiotics in food products such as inulin for use in a weight management or attenuation program. More specifically, the use inulin or its natural constituent(s) in food products increases the uptake of calcium, in certain or select segments or demographics of the population providing beneficial weight management effects. The present invention relates to the use of such a food product in a weight management or attenuation program in a select demographic sector of the population as well as the communication of the health benefit of that product to the demographic.

### CROSS-REFERENCES TO RELATED APPLICATIONS

None.

### BACKGROUND OF THE INVENTION

The present invention relates to the inclusion of certain prebiotics in food products, such as inulin, for use in a weight management or attenuation program. More specifically, the use inulin or its natural constituent(s) in food products increases the uptake of calcium, in certain or select segments or demographics of the population. The present invention relates to the use of such a food product in a weight management or attenuation program in a select demographic sector of the population as well as the communication of the health benefit of that product to the demographic. The use of calcium in addition to a number of other benefits has been shown in some cases to prevent the formation of fatty adipose tissue thereby permitting the host greater weight management control.

Increasing the level of calcium in one's diet has become increasingly important over the years in that calcium is known to provide a number of benefits such as enhancing bone mineral density to aid in the prevention of osteoporosis.

U.S. Pat. No. 6,384,087 relates to materials and methods for treating obesity in humans through the use of a diet that is high in calcium. Such a suggested diet includes increased levels of dairy products, dietary or vitamin supplements or other sources of calcium such as calcium carbonate. However, while potentially effective, consumers may not wish to ingest a high level of dairy products or take supplements in order to obtain the benefits of a diet high in calcium. Moreover, certain segments of the population may be unable to participate in a high dairy diet.

A number of other research efforts relating to the effect of dietary calcium on body weight and fat have been produced and include the use of dietary intake of calcium being negatively associated with body mass in adults McCarron et al. Blood Pressure and Nutrient Intake in the United States Science, 1984 224:1392-1398.

Dietary intake of calcium is negatively associated with children's fat mass (ages 24 to 60 months) Carruth et al. Dietary and Anthropometric Factors Predicting Body Fat in Preschool Children. Scand. J. Nutr. 1999: 43 (Supp. 34): 53S.

Davies et al. J. Clin. Endocrinol. Metab., 2000; 85:4635-4638 provides and analysis of several studies that looked at the effect of calcium supplementation on bone health in women of various ages from the standpoint of effect on body weight yields an estimate that a 1000-mg calcium intake increase is associated with an 8-kg decrease in mean body weight.

Lin et al. Dairy Calcium is Related to Changes in Body Composition During a Two-Year Exercise Invervention in Young Women J. Amer. Coll. Nutr. 2000; 19:754-760 provided an analysis that included a group of young women (18-31 years of age) that was provided with a high calcium intake, adjusted for total energy intake, gained less weight and body fat than those who had low calcium intake.

Chan and McNaught The Effects of Dairy Products on Children's Body Fat J. Amer. Coll. Nutr. 2001; 20:577 illustrated that children (ages 2.5 to 8.8 years of age) that had diets supplemented with 4 servings of dairy foods per day, thereby increasing their calcium intake from approximately 880 mg to 12000 mg calcium per day, gained less body fat over the course of a 6 month study (0.3 kg vs. 1.5 kg) than children whose diets are otherwise similar (calories, fat, carbohydrates) though height and weight gains were similar.

Other studies have been provided which indicate that inulin may increase the level of calcium absorption in the body. However, in certain circumstances, it has been shown that select portions of the population may not benefit from the presence of inulin in the diet as it relates to calcium absorption.

Publications, patents and patent applications are referred to throughout this disclosure. All references cited herein are hereby incorporated by reference.

What is needed therefore is a food product that is part of a weight management program that will facilitate weight attenuation in select demographics of the population and which does not require modifying one's dietary patterns in order to obtain the suggested benefit.

### BRIEF SUMMARY OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

In one embodiment of the present invention a food product for use in connection with a weight management program for a select demographic is described and includes a collection of macronutrients and micronutrients that are beneficial to maintaining a healthy diet. At least one of the micronutrients is calcium. The food product also includes a source of prebiotic. A predefined message providing information relating to benefits of including the food product as part of a diet ranging from 1000 to 2500 calories. The macronutrients and micronutrients are mixed along with the source of prebiotic into a food intermediate to form said food product useable in the weight management program.

In a further embodiment of the present invention a method for communicating a weight management program to a select demographic is described and includes the steps of initially providing a food product having at least a source of calcium and a source of inulin. The source of calcium amounts to 100% or less than a recommended daily allowance of calcium. The communication includes the description of a diet plan that includes the food product and indicates the recommended serving sizes of the food product. The method includes communicating the diet plan and its weight management benefits through a prearranged media format.

A still further embodiment of the present invention relates to a weight attenuation message intended for use in a selected demographic portion of society, the message includes a food product that has a pre-established grouping of micronutrients and macronutrients; a source of calcium and a soluble dietary fiber provided at least as part of the macronutrients and micronutrients. The food product also includes a source of prebiotic combined with the food product. A message provided in a preselected format including print, audio and visual. The message is intended to communicate a health benefit of the food product that has a source of prebiotic, calcium and soluble dietary fiber. The health benefit of the message is related to weight attenuation in a selected demographic.

The food product and the method of communicating the food product as part of a weight management program are targeted at specific demographics, such as age related demographics or larger groupings of demographics such as those segments of the population considered to be proactive in looking for health benefits.

The communication of the weight management message can be done through a number of media outlets and includes such things as printed indicia on product packages, printed messages in magazines, newspapers, newsletters and the like, audio and visual outlets such as radio and television as well as other outlets suitable for use in delivering the message to the targeted demographic.

These, as well as other objects and advantages of this invention, will be more completely understood and appreciated by referring to the following more detailed description of the presently preferred exemplary embodiments of the invention.

### DETAILED DESCRIPTION OF THE INVENTION

The marketplace today is filled with a variety of food products, food intermediates, ingredients and the like that are intended for human and/or animal consumption and directed at a particular health attribute. Often the difficulty in introducing such products to the marketplace surrounds the manner through which the product or intermediate is communicated to the marketplace. In order to effectively introduce a product, the producer or manufacturer needs to target the product at a particular audience or demographic.

In order to develop a targeted message, research is conducted to determine those segments of society that are likely to purchase the product, specifically those intending to receive the health benefit of the product. Certain demographics or population segments may include age groupings, such as those above 55 years of age or those in the 35 to 54 age category, or a mixture of those groupings who may be proactive in a attaining a greater health benefit.

Testing to determine the relevant demographic or demographics is usually through a variety of methods. This testing may include but is not limited to product or concept testing, consumer test panels, in home tests or random testing done in high traffic areas, such as shopping malls, or even testing done over the Internet. Once the testing is completed, the product concept is further developed and refined and the associated health benefit message is created.

In determining the appropriate demographic portion of the population, the present invention may use various databases that provide information that may have been created internally or obtained on a subscription or contract basis from third party suppliers. For example, it is possible to purchase or license useful data sets from a variety of sources including for example A. C. Nielsen, Spectra Marketing, and others. For example, A. C. Nielsen provides so-called consumer panel data that supplies consumer purchase information based on diaries and the like. A. C. Nielsen also provides SCANTRACK and Market Dimension data sets that track consumer purchases in a given market through data collection based on in-store checkout scanners. Spectra Marketing provides demographic-based consumer information that can be used to develop sales and in-store marketing strategies.

The present invention is directed to a food product, such as a ready to eat (RTE) cereal, RTE meal, snacks, bakery products, baking mixes, dough based products, such as breads, rolls and the like, snack bars and fruit based confections, cereal bars, food served “on the go”, yogurt based product and other convenience foods. The food product provided herein includes a predefined grouping of macronutrients and micronutrients of which a source of calcium and soluble dietary fiber is included.

The macronutrients and micronutrients suitable for use in the present invention include but are not limited to: fat; carbohydrates; protein; vitamin A; Vitamin C; Vitamin D; Vitamin E; Vitamin K; Thiamine; riboflavin; niacin; Bitamin B6; folate; Vitamin B 12; biotin; Pantothenic Acid; calcium; potassium; iron; phosphorus; iodine; magnesium; selenium; copper; manganese; chromium; molybedenum; and zinc. In addition, the composition of the food product may also include various antioxidants, phytochemicals; nutraceuticals and non-nutritive elements such as food coloring, emulsifiers, flavoring agents, etc.

In addition, the food product of the present invention is provided with a source of inulin which has the ability to increase calcium absorption in certain segments of the population, for example adults over the age of 35.

Inulin is found in a wide variety of plant material and is present in significant amounts in vegetables such as artichokes, asparagus, leeks, onions, garlic, chicory roots as well as a number of other sources. Commercial sources of inulin are marketed under the names RAFTILINE® and RAFTILOSE® and are available from Orafti, Malvern, Pa. RAFTILINE® comprises approximately 92% by weight inulin and RAFTILOSE® comprises about 100% by weight inulin. Inulin and is a soluble dietary fiber and may provide some benefit in reducing cholesterol levels.

A number of prebiotics may be used in connection with inulin and have been identified as suitable for use in increasing calcium levels in the diet. A prebiotic is a non-digestible ingredient (that is suitable for human and/or animal consumption), which provides beneficial effects by stimulating select bacteria in the colon in order to improve the health of the host.

For an ingredient to qualify as a prebiotic it cannot be hydrolyzed or absorbed completely in the upper part of the gastrointestinal tract and must be able to stimulate or activate select bacteria, make a beneficial adjustment of the colonic flora and induce certain effects that are healthful for the host ingesting the ingredient.

Possible prebiotics for use in the present invention include but are not limited to the following: fructo-oligosaccharides (inulin obtained from chicory root); galacto-oliosaccharides; soybean oligosacchardies (obtained from soybean whey); gluco-oligosaccharides (such as isomalto-oligoaccharides); xylo-oligosaccharides; lactosucrose; medium to high amylose starches (such as Hylon V a high amylose, food grade starch having greater than 50% amylose, is derived from corn and is available from National Starch of Bridgewater, N.J.); soluble fibers (beta glucan); insoluble fibers; short chain fatty acids; D-tagatose; Lactulose; sugar alcohols (e.g. lactitol, maltitol, erythritol); lacto-fructo-oligosaccharides; polydextrins; gums (e.g. guar gum, xanthan gum, gum Arabic); pectin; psyllium; arabinogalactan; arabinoxylan.

In one embodiment of the present invention the preferred prebiotic is fructo-oligosacchardies which is a known and acceptable food ingredient. The prebiotics may be used alone or in combination with one another.

One exemplary source of soluble fiber is beta glucan a suitable prebiotic, that can be obtained from a number of grains or cereals, such as oat, wheat, corn, rice and barley. Two of the more preferred sources of beta glucan come from oat bran and barley. Beta glucan is a major polysaccharide of oat and barley, and is made of linear polymers composed of glucosyl residues linked via a mixture of β-(1→14) and β-(1→3) linkages in a ratio of about 2.3-3.0 to 1.0 (Izydorcyk et al., Journal of Cereal Science 27, pp. 321-325, 1998 and Welch Id).

Oat bran used in the present invention is produced by grinding clean oat groats or rolled oats and separating the resulting flour by suitable means, such as sieving, into fractions such that the oat bran fraction is not more than 50% of the original starting material. The separated fraction should have at least 5.5% of beta glucan (dry weight basis), and a total dietary fiber content of at least 16% (dry weight basis), so that at least one third of the total dietary fiber is soluble fiber.

Barley, as used in the present invention, is processed in a manner that resembles oats as set forth above, in that it consists of cleaning, hulling, sieving and then grinding. Waxy hulless barley has a higher dietary fiber content than most other sources of fiber and can range from 14 to 20% of the dry weight and have a beta glucan content of around 8 to 10%.

Beta glucan coexists in the cell with β-glucanases, which are enzymes that break down beta glucan. β-glucanases are released or activated when a viable grain is hydrated. Additionally, as the grains germinate, newly made β-glucanase is released from the scutellum and from the grain aleurone layer into the endosperm. Thus, as water is added to the grain, beta glucan is degraded and broken down into smaller units through the activity of β-glucanase. For example, extracted beta glucan can be treated with hydrolytic enzymes, such as laminarinase or β(1→3) glucanases, to reduce the degree of β(1→3) linkages of the extracted beta glucan, which results in a decrease in the solution viscosity profile.

The present invention is now illustrated in greater detail by way of the following examples, but it should be understood that the present invention is not to be construed as being limited thereto.

### EXAMPLE 1

In this example the nutritionally complete food product, in this case a ready to eat (RTE) cereal, may include the following micronutrients and macronutrients in connection with a source of inulin or suitable prebiotic ranging from 0.5 to 9 grams of inulin. The serving size ranges from 30 to 55 grams.

Recommended Daily Value (“DV”)

| | |
|---|---|
| Total Fat | 4% |
| Saturated Fat | 3% |
| Cholesterol | 0% |
| Sodium | 10% |
| Potassium | 5% |
| Total Carbohydrate | 14% |
| Dietary Fiber | 17% |
| Protein | 10% |
| Vitamin A | 10% |
| Vitamin C | 70% |
| Calcium | 100% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products baking mixes and ready to eat meals may contain additional vitamins, nutrients and or minerals as well as potentially varying amounts of the macro and micro nutrients set forth in the instant example. In addition, where the diet represents a 2500-calorie diet the proportional amount of the ingredients may be increased as is necessary. The invention is suitable for use with diets ranging from 1000 calories to 2500 calories.

The RTE cereal of the preceding example is prepared in a conventional manner. This exemplary RTE cereal is in the form of flakes that are created by preparing a cooked cereal dough through known methods and then forming the cooked cereal dough into pellets that have a desired moisture content. The pellets are then formed into wet flakes by passing the pellets through chilled roller and then subsequently toasting or heating the wet cereal flakes. The toasting causes a final drying of the wet flakes, resulting in slightly expanded and crisp RTE cereal flakes. The flakes are then screened for size uniformity. The final flake cereal attributes of appearance, flavor, texture, inter alia, are all affected by the selection and practice of the steps employed in their methods of preparation. For example, to provide flake cereals having a desired appearance feature of grain bits appearing on the flakes, one approach is to topically apply the grain bits onto the surface of the flake as part of a coating that is applied after toasting.

In one double blind study ranging over a period of three weeks, a number of adolescent girls, ages 11-13, were provided with a diet containing roughly 1600 mg/calcium per day and 9 grams of inulin. The diet was delivered as part of a RTE cereal and the amount of calcium retention and absorption was measured by mass balance and stable isotope enrichment. Stool and urine samples were collected and analyzed for calcium excretion by atomic absorption spectrometry. Calcium retention was calculated as: Diet Ca−urine Ca−fecal Ca. Blood samples were also taken at prescribed intervals and serum and urine were analyzed for Ca by plasma mass spectrometry. The results of the analysis provided that inulin had no effect on the retention or absorption of calcium by subjects in this particular demographic.

Applicants' postulate that the disappointing result was obtained in that the metabolism of the subjects was already running at optimum levels, thereby prohibiting the additional uptake of inulin and calcium. However, food products prepared in accordance with the present invention have shown beneficial results with other segments of the population where the metabolism of the subject was operating at different levels of efficiency or those portions of the populations that suffer from other deficiencies, such as subjects that are lactose sensitive or intolerant. Those examples include teenage boys which showed an average increase of approximately 26% in calcium absorption and a 58% improvement of calcium absorption in adults, ages 21 and above and more specifically the groups 35-54 years of age and 55 and older.

In addition, those particular demographics that consume higher levels of dairy products may also find the present invention useful in increasing the overall level of calcium retained by the body itself.

It is believed that food products prepared in accordance with the present invention, would however provide an enhanced benefit to certain selected demographic sections of the population, namely postmenopausal women. These results suggested that in particular, postmenopausal women, whose calcium absorption machinery is markedly reduced in efficiency may benefit from the presence of inulin in a food product as part of a weight attenuation program in that the level of calcium absorbed and retained was higher than in the placebo portion of the study.

The food product of the present invention, while providing well balanced nutrition for consumers, is intended for use in connection with a certain demographic segment of the population that will benefit more fully from the offering.

It will thus be seen according to the present invention a highly advantageous food product for use in weight attenuation has been provided. While the invention has been described in connection with what is presently considered to be the most practical and preferred embodiment, it will be apparent to those of ordinary skill in the art that the invention is not to be limited to the disclosed embodiment, that many modifications and equivalent arrangements may be made thereof within the scope of the invention, which scope is to be accorded the broadest interpretation of the appended claims so as to encompass all equivalent structures and products.

1. A food product for use in connection with a weight management program for a select demographic, said food product comprising,
a collection of macronutrients and micronutrients beneficial to maintaining a healthy diet at least one of said micronutrients is calcium;
a source of prebiotic;
a predefined message providing information relating to benefits of including said food product as part of a diet ranging from 1000 to 2500 calories; and
wherein said macronutrients and said micronutrients are mixed along with said source of prebiotic into a food intermediate to form said food product useable in the weight management program.

2. A food product for use in connection with a weight management program as recited in claim 1, wherein said select demographic includes a first group.

3. A food product for use in connection with a weight management program as recited in claim 2, wherein said first group are adults between ages of 35-54.

4. A food product for use in connection with a weight management program as recited in claim 1, wherein said select demographic includes a first group and a second group.

5. A food product for use in connection with a weight management program as recited in claim 4, wherein said second group are adults above age 55.

6. A food product for use in connection with a weight management program as recited in claim 4, wherein said select demographic includes a ratio of said first group to said second group.

7. A food product for use in connection with a weight management program as recited in claim 6, wherein said ratio ranges from approximately less than 50% of said first group to approximately greater than 50% of said second group.

8. A food product for use in connection with a weight management program as recited in claim 6, wherein said ratio ranges from approximately 20-30% of said first group to approximately 70-80% of said second group

9. A food product for use in connection with a weight management program as recited in claim 1, wherein said select demographic includes adults that are proactive, health conscious individuals.

10. A food product for use in connection with a weight management program as recited in a claim 1, wherein said food product includes a health benefit message provided in a preselected format.

11. A food product for use in connection with a weight management program as recited in a claim 1, wherein said prebiotic is selected from the group consisting of fructo-oligosaccharides; galacto-oliosaccharides; soybean oligosacchardies; gluco-oligosaccharides; xylo-oligosaccharides; lactosucrose; medium to high amylose starches; soluble fibers; insoluble fibers; short chain fatty acids; D-tagatose; Lactulose; sugar alcohols; lacto-fructo-oligosaccharides; polydextrins; gums; pectin; psyllium; arabinogalactan; arabinoxylan.

12. A food product for use in connection with a weight management program as recited in a claim 1, wherein said prebiotic is inulin.

13. A method of communicating a weight management program for a select demographic comprising the steps of;
providing a food product having at least a source of calcium and a source of inulin, said source of calcium amounts to 100% or less than a recommended daily allowance of calcium;
describing a diet plan including said food product and recommended serving sizes of said food product; and
communicating said diet plan and its weight management benefits through a prearranged media format.

14. A method of communicating a weight management program as recited in claim 13, wherein said select demographic includes a first group and a second group.

15. A method of communicating a weight management program as recited in claim 14, wherein said first group are adults between ages of 35-54.

16. A method of communicating a weight management program as recited in claim 14, wherein said second group are adults above age 55.

17. A method of communicating a weight management program as recited in claim 13, including a further step of providing a source of soluble dietary fiber.

18. A weight attenuation message intended for use in a selected demographic portion of society, comprising;
a food product, said food product having a pre-established grouping of micronutrients and macronutrients;
a source of calcium and a soluble dietary fiber provided at least as part of said macronutrients and said micronutrients;
a source of prebiotic combined with said food product;
a message provided in a preselected format, said format including print, audio and visual;
said message communicates a health benefit of said food product having said source of prebiotic, said source of calcium and said soluble dietary fiber; and
wherein said health benefit of said message is related to weight attenuation in a selected demographic.

19. A weight attenuation message as recited in claim 18, wherein said selected demographic includes a first group.

20. A weight attenuation message as recited in claim 18, wherein said first group are adults between ages of 35-54.

21. A weight attenuation message as recited in claim 18, wherein said select demographic includes a first group and a second group.

22. A weight attenuation message as recited in claim 21, wherein said second group are adults above age 55.

23. A weight attenuation message as recited in claim 18, wherein said soluble dietary fiber is beta glucan.

24. A weight attenuation message as recited in claim 18, wherein said select demographic includes lactose sensitive people.

25. A weight attenuation message as recited in claim 18, wherein said select demographic includes postmenopausal women.

26. A weight attenuation message as recited in a claim 18, wherein said prebiotic is selected from the group consisting of fructo-oligosaccharides; galacto-oliosaccharides; soybean oligosacchardies; gluco-oligosaccharides; xylo-oligosaccharides; lactosucrose; medium to high amylose starches; soluble fibers; insoluble fibers; short chain fatty acids; D-tagatose; Lactulose; sugar alcohols; lacto-fructo-oligosaccharides; polydextrins; gums; pectin; psyllium; arabinogalactan; arabinoxylan.

27. A weight attenuation message as recited in a claim 18, wherein said prebiotic is inulin.
